(19) United States
(12) Patent Application Publication
(10) Pub. No.: US 2008/0208096 A1
(43) Pub. Date: Aug. 28, 2008

(54) Mobility Aids

(76) Inventor: Kenneth Rayvenor Lusty Armitage, Devon (GB)

Correspondence Address: MELVIN I. STOLTZ, ESQ., 51 CHERRY STREET, MILFORD, CT, 06460, US

(21) Appl. No.: 11/884,647

(22) PCT Filed: Feb. 17, 2006
(86) PCT No.: PCT/GB2006/000554

§ 371 Date: Aug. 17, 2007

(30) Foreign Application Priority Data
Feb. 17, 2005 (GB) 0503323.8

(51) Int. Cl. A61F 5/00

(52) U.S. Cl. 602/28

(57) ABSTRACT

A mobility aid includes a harness that can be placed on the upper body of the user, an elasticated cord that can be attached to a foot of the user, and means for attachment of the elasticated cord to the harness.

### FIELD OF THE INVENTION

This invention relates to mobility aids and is concerned with the provision of an improved form of aid.

Humans walk on two legs, on feet that have to be raised and lowered on each step forward and the relevant muscles are instructed by nerve communication to move the legs relative to the hips and the feet relative to the legs.

There are muscles in the front of the lower leg that serve to raise the foot and to decrease or reduce the angle between the leg and the foot. There are other muscles in the lower leg that serve to move the foot angle down relative to the leg and to govern the plane of movement of the foot relative to the leg. There are muscles in the hip and upper leg to move the leg in a fore and aft plane and to control movements into and out of that plane. Some leg and hip muscles are instructed by nerve communication to move the foot relative to the leg as the leg is moved forward or raised for each step.

The above articulations and movements can be disabled by muscle or nerve damage and common ailments can cause such disabilities. In particular, persons suffering from sciatica or multiple sclerosis or who have had strokes can have the above disabilities.

Even though the primary leg-straightening muscles of a person may be able to deliver tens of kilograms of pressure, that person may be disabled or impeded because other muscles cannot be instructed to deliver lesser pressures to permit walking or climbing stairs.

It is accordingly an object of the present invention to provide an improved form of mobility aid.

It is also an object of the present invention to provide a method of improving the walking capability of a disabled person.

### SUMMARY OF THE INVENTION

According to a first aspect of the present invention there is provided a mobility aid that includes a harness that can be placed on the upper body of the user, an elasticated cord that can be attached to a foot of the user, and means for attachment of the elasticated cord to the harness.

The aid may include a single elasticated cord, or it may include two elasticated cords, one for attachment to each foot of the user.

The harness preferably includes adjustment means.

The means for attachment of the elasticated cord to the harness preferably also includes adjustment means for adjustment of the position of the upper end of the elasticated cord.

Means are preferably also provided for adjusting the length of the elasticated cord and thus the tension in the elasticated cord.

According to a second aspect of the present invention there is provided a method of improving the walking capability of a disabled person that includes providing a harness and an elasticated cord, placing the harness on the upper body of the disabled person, attaching the elasticated cord to the harness, and attaching the elasticated cord to a foot of the disabled person.

### BRIEF DESCRIPTION OF THE DRAWINGS

FIG. 1 is a schematic front view of a person having a basic leg support harness,

FIG. 2 is a schematic rear view of the person shown in FIG. 1,

FIG. 3 is a schematic front view of a person having a standard harness,

FIG. 4 is a schematic rear view of the person shown in FIG. 3,

FIG. 5 is a schematic front view of a person having a full harness,

FIG. 6 is a schematic rear view of the person shown in FIG. 5, and

FIG. 7 is a schematic detail view showing the attachment of an elasticated cord to a foot.

### DESCRIPTION OF THE PREFERRED EMBODIMENTS

The drawings show a number of embodiments of the invention, each of which includes a shoulder harness 10 or a body harness 11, one or more elasticated assemblies 12 and one or more clips or connectors 13, such as that shown in FIG. 7, for connecting the lower end of the associated elasticated assembly 12 to the foot of the user. The or each elasticated assembly 12 includes an elasticated cord able to withstand a significant tension and an appropriate covering.

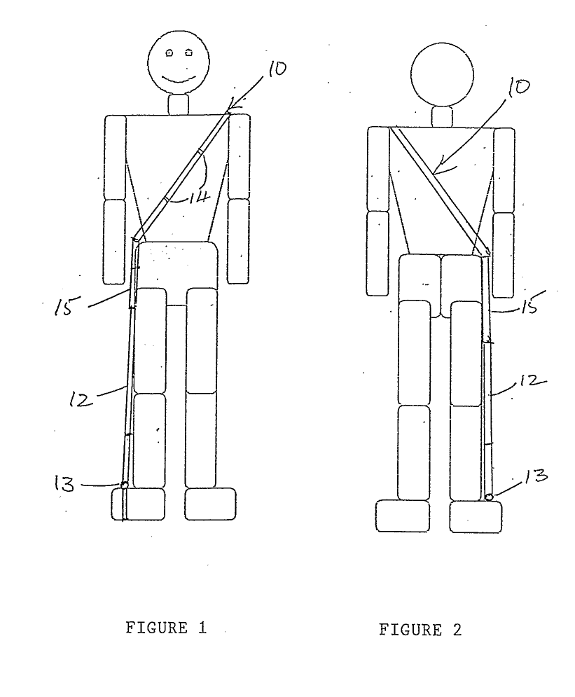

The shoulder harness 10 shown in FIGS. 1 and 2 includes a loop of webbing that is adjustable in length by means of a pair of spaced buckles 14. For a person whose right leg is disabled, the shoulder harness 10 is placed over the head and the right shoulder, as shown, ensuring that the buckles 14 are on the chest of the user, in front of the body so as to permit ready adjustment of the harness 10 so that the joint between the front and the rear straps forming the harness is on the hip of the person fitted with the harness 10.

A foamed shoulder pad may be placed on the left shoulder of the user of the mobility aid to provide a cushioning effect and to spread the load applied to the shoulder.

The harness 10 is connected to the elasticated assembly 12 by means of a strap 15 that is adjustable in length using, for example, a square ring length adjuster. Adjustment of the length of the strap 15 varies the height to which the right leg of the user will be lifted by means of the aid. The strap 15 may also include a quick-release mechanism for extending the length of the strap 15 and thus reducing the tension in the elasticated assembly 12 should this reduction in tension be required for any reason. Conversely, the tension can be increased for changes in gradient, for climbing stairs or other similar activities.

The elasticated assembly 12 is provided at its upper end with a clip by which it is connected to a fitting at the lower end of the strap 15 and there is a loop of adjustable size at the lower end of the elasticated assembly 12. Varying the size of the loop and thus the relaxed length of the elasticated assembly 12 will vary the tension to which the assembly 12 is subjected in use and thus the force applied to the foot of the user. The material from which the elasticated assembly 12 is produced is typically such that it is stretched by between 15 and 20% of its length when taking the weight of each limb.

The aid may include a shoe harness that can be fitted around the shoe of the user. The harness is opened and placed under the shoe. The shoe harness includes straps that fit over the shoe and around the back of the shoe. A clip 13 is provided at the lower end of the elasticated assembly and the clip 13 is attached to the shoe harness once the shoe harness has been attached securely to the shoe of the user.

The clip 13 at the lower end of the elasticated assembly 12 can alternatively be attached to the shoelaces of the shoe on the foot of the user, as shown in FIG. 9, the shoelaces being tied securely around a portion of the clip 13 to provide a secure attachment of the lower end of the elasticated assembly 12 to the shoe.

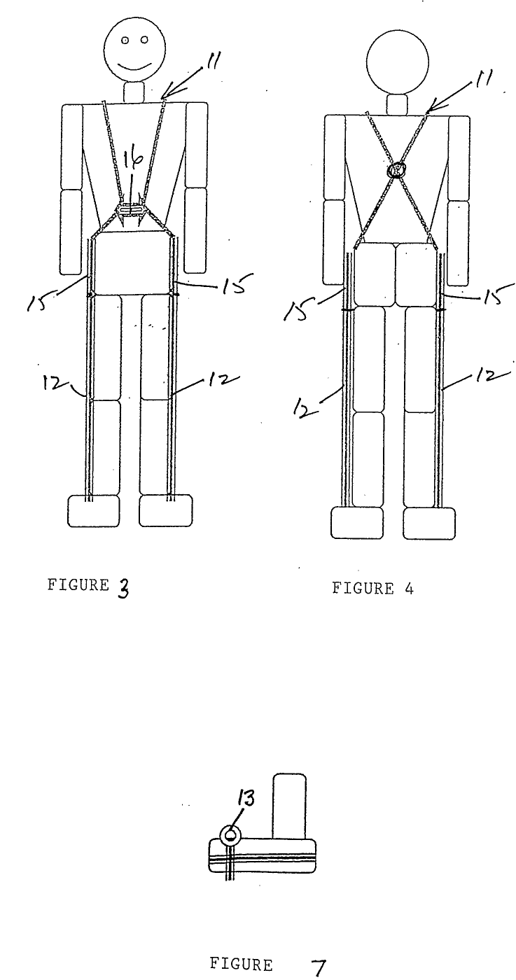

The mobility aid shown in FIGS. 3 and 4 differs from that shown in FIGS. 1 and 2 in that the body harness 11 that includes straps that pass over both shoulders of the user and the straps are joined together by means of a fitting 16 that permits adjustment of the lengths of the straps so that the harness 11 can be attached securely and comfortably to the upper body of the user. There are then two elasticated assemblies 12, one for attachment to each foot of the user, and two straps 15, one for attaching each of the elasticated assemblies 12 to the harness 11. The lengths of the straps 15 and the sizes of the loops in the elasticated assemblies will be adjusted as appropriate to the requirements of the user.

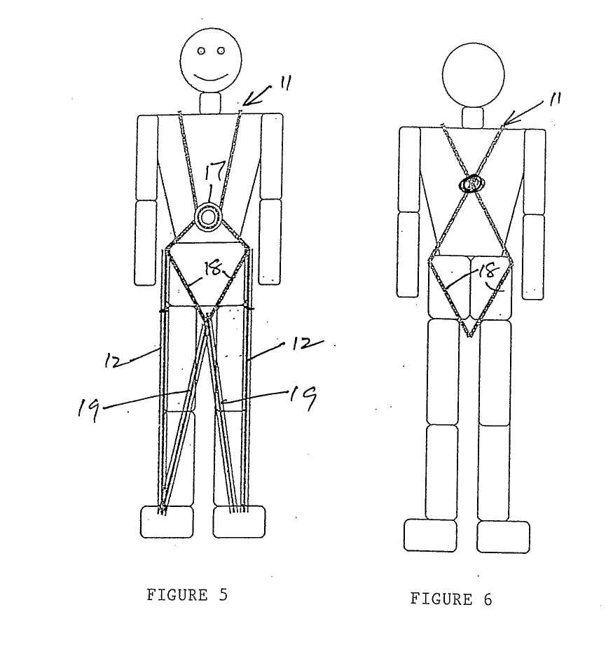

The mobility aid shown in FIGS. 5 and 6 differs from that shown in FIGS. 3 and 4 in that the body harness 11 is a full body harness that is configured to minimise out-of-plane joint action. It includes a central strap connector 17, additional straps 18 and additional elasticated assemblies 19. The lengths of the additional straps 18 and of the additional elasticated assemblies 19 are adjustable to fit the requirements of the user.

Other harness configurations can be used in addition to the specific forms shown in the drawings.

Multiple sclerosis affects the ability of the body to transmit instructions by nerve signals, particularly to weaker muscles in peripheral places, such as muscles for moving limbs. A person suffering from multiple sclerosis may be limited to walking 200 meters in about ten minutes each day and, although the large push-down muscles that put most of the effort into walking may not be significantly disabled by the multiple sclerosis, such muscles can become disabled as a result of years of disuse.

Use of one of the aids shown in the drawings will enable a person suffering from multiple sclerosis or from any of the other disabilities described above to become much fitter by expending more energy at a higher rate. Thus, use of an aid may enable a person suffering from multiple sclerosis to walk for, for example, thirty minutes and cover a distance of the order of 1.5 kilometres in that time. It is to be noted that the body performs work, such as walking, at a rate that is limited by the capability of the weakest part of the body used in that work. Use of the aid will thus enable a person suffering from multiple sclerosis to walk for longer distances and at a faster rate than was possible without the aid. There will also be a significant improvement in the overall walking ability of the user such that after, for example, utilising the aid for regular walking for a month, the user will be able to walker faster and for longer even without the aid.

As a result of tests that have been carried out on a number of disabled people, an average initial improvement of 42% was obtained in respect of the walking speed using the aid as compared to not using the aid. If the aid was then used regularly for two months an increase in the walking speed obtained without the aid was registered. This was an average of 28% for one month's use of the aid and 38% after two months' use of the aid.

1. A mobility aid that includes a harness that can be placed on the upper body of the user, an elasticated cord that can be attached to a foot of the user, and means for attachment of the elasticated cord to the harness.

2. A mobility aid as claimed in claim 1, which includes a single elasticated cord.

3. A mobility aid as claimed in claim 1, which includes two elasticated cords, one for attachment to each foot of the user.

4. A mobility aid as claimed in claim 3, in which the harness includes adjustment means.

5. A mobility aid as claimed in claim 4, in which the means for attachment of the elasticated cord to the harness includes adjustment means for adjustment of the position of the upper end of the elasticated cord.

6. A mobility aid as claimed in claim 5, in which means are provided for adjusting the length of the elasticated cord and thus the tension of the elasticated cord.

7. A method of improving the walking capability of a disabled person that includes providing a harness and an elasticated cord, placing the harness on the upper body of the disabled person, attaching the elasticated cord to the harness, and attaching the elasticated cord to a foot of the disabled person.

8. A method as claimed in claim 7, which includes adjusting the position of the upper end of the cord relative to the harness.

9. A method as claimed in claim 8, which includes adjusting the length of the elasticated cord and thus the tension in the elasticated cord.
